(19) United States
(12) Patent Application Publication
(10) Pub. No.: US 2020/0286618 A1
(43) Pub. Date: Sep. 10, 2020

(54) BEHAVIOUR CAPTURE DEVICE

(71) Applicant: OLITOOL LTD, London (GB)

(72) Inventors: Elizabeth Ann ERICKSON, London (GB); Deborah WERBNER, London (GB); Lawrence ARCHARD, Lidlington (GB); Kyoko MURAKAMI, Copenhagen (DK)

(73) Assignee: OLITOOL LTD, London (GB)

(21) Appl. No.: 16/761,274

(22) PCT Filed: Oct. 31, 2018
(86) PCT No.: PCT/GB2018/053159

§ 371 Date: May 4, 2020

(30) Foreign Application Priority Data
Nov. 3, 2017 (GB) 1718248.6

(51) Int. Cl. G16H 40/63

(52) U.S. Cl. CPC G16H 40/63

(57) ABSTRACT

A handheld or wearable behaviour capture device (100) comprises a physical input interface (101) configured to receive an input from a user (110) related to the user's behaviour. The input interface (101) is used to generate user input data. The device (110) further comprises a processor (102) configured to: receive the user input data; determine if the user input data corresponds to a plurality of predetermined identifiers; and generate feedback data. A feedback module (104) is provided and configured to receive the feedback data from the processor (102) and to provide feedback to the user (110).

### TECHNICAL FIELD

This invention relates to a device and method for capturing the behaviour of a user and for managing behavioural health.

### BACKGROUND TO THE INVENTION AND PRIOR ART

Individuals of any age may choose to monitor their behaviours, emotions, attitudes, actions, habits, moods and feelings, etc. (hereinafter referred to collectively “behaviour(s)”) in order to help them achieve certain health, wellbeing, emotional or lifestyle goals. Mental and behavioural health challenges, including anxiety, depression, addictions, smoking, weight management, persistent pain and childhood emotional and behavioural issues such as Autism Spectrum Disorder (ASD) and Attention Deficit Hyperactivity Disorder (ADHD), learning disabilities, speech and language or other communication disorders, social and emotional learning challenges, can be addressed, in part, by well-established psychological approaches, including Cognitive Behaviour Therapy (CBT) and behaviour change techniques. These approaches aim to support individuals attain health, wellbeing or lifestyle goals by amongst other things observing, tracking, identifying, and analysing the individual's behaviours and other information related to the individual's behavioural health, emotional, psychological and/or physical states over a period of time. Gathering and analysing this information allows the individual and/or therapist to assess the behaviours in question, develop an understanding of behaviour triggers, set targets for behaviour change and offer relevant support and resources.

Hence, monitoring relevant behaviours is vital for implementing these psychological and behaviour change approaches to meet health, wellbeing and/or lifestyle goals. Traditional methods of behaviour monitoring involve the active maintenance by an individual of a written log or diary of his or her behaviours, psychological and/or physical state. A written diary or log may be impractical and laborious, challenging the users' engagement with the approach, or inappropriate in the case of young children or others with language or communication challenges.

Other monitoring methods include sensors on wearable devices that passively record physiological information, such as heart rate and galvanic skin response (GSR), to infer psychological and/or physical states. However, such methods do not capture behavioural and cognitive elements or encourage user engagement, and the information about psychological and/or physical states may be limited by the type of sensors and the nature and accuracy of the inference.

There is therefore a need for an improved, more inclusive and engaging behaviour tracking tool to aid individuals of all ages reach their health, wellbeing and lifestyle goals.

### SUMMARY OF THE INVENTION

According to a first aspect of the invention there is provided a handheld or wearable behaviour capture device comprising:
a physical input interface configured to receive input from a user related to the user's behaviour, and to generate user input data;
a processor configured to:
   receive the user input data;
   determine if the user input data corresponds to at least one of plurality of predetermined identifiers; and
   generate feedback data; and
a feedback module configured to receive the feedback data from the processor and to provide feedback to the user.

The physical act of a user in manipulating the behaviour capture device corresponds with either an action to intentionally indicate or communicate pre-assigned behaviours (e.g. moods, thoughts, feelings, emotions, actions, habits, personal or collective memories, behaviours or other information related to the users psychological, emotional, physiological or behavioural health) or more broadly a means to distract, self-soothe, and discharge emotions, energy, feelings, concerns, etc. otherwise known as fidgeting.

The device may further comprise memory, wherein the processor may be further configured to store a bookmark record in the memory in response to determining that the user input data corresponds to one of the predetermined identifiers.

The processor may be further configured to store a non-bookmark record, including fidgeting activity, in the memory in response to determining that the user input data does not correspond to one of the predetermined identifiers.

The record may include the predetermined identifier and one or more of a time, a duration, an amount of pressure and a location of the user input.

The predetermined identifier may be a pattern corresponding to a plurality of distinct user inputs of predetermined duration.

The feedback module may comprise one or more of a haptic feedback motor, a display and illumination means.

The physical input interface may be chosen from one or more of a force sensor, a pressure sensor, a push button and/or a switch configured to detect one or both of a force or a pressure applied to the physical input interface and/or an accelerometer, gyroscope, magnetometer or sensor to detect motion.

The device may further comprise sensing means configured to generate sensing data indicative of the orientation and acceleration forces applied to the behaviour capture device.

The device may further comprise a GNSS receiver.

The device may further comprise a transceiver.

According to a second aspect of the invention there is provided a handheld or wearable behaviour capture device comprising:
a resiliently deformable input interface for generating user input data;
a processor configured to:
   receive the user input data from the input interface;
   generate feedback data; and
a feedback module configured to receive the feedback data from the processor and to provide feedback to the user.

The device may further comprise memory, wherein the processor may store a bookmark record in the memory in response to determining that the user input data corresponds to a predetermined identifier.

The processor may be further configured to store a non-bookmark record, including fidgeting activity, in the memory in response to determining that the user input data does not correspond to a predetermined identifier.

The record may include one or more of an amount of pressure, time, duration, intensity and location of the user input.

The predetermined identifier may correspond to a plurality of distinct user inputs of predetermined duration.

The feedback module may comprise one or more of a haptic feedback motor, a display and illumination means.

The physical input interface may be chosen from one or more of a force sensor, a pressure sensor, a push button and a switch configured to detect one or both of a force or a pressure applied to the input interface.

The device may further comprise sensing means configured to generate sensing data indicative of the orientation and acceleration forces applied to the behaviour capture device.

The device may further comprise a GNSS receiver.

The device may further comprise a transceiver.

According to a further aspect of the invention there is provided a behaviour capture system comprising: a behaviour capture device as described above and a computing device configured to communicate with the transceiver.

The computing device may be one or more of a mobile phone, tablet device, laptop, notebook or server.

According to a further aspect of the invention there is provided a method for behaviour capture comprising:
providing a behaviour capture device as described above;
receiving at the input interface an input from a user related to the user's behaviour and generating user input data;
receiving the user input data at the processor;
determining at the processor if the user input data corresponds to one of a plurality of predetermined identifiers;
generating at the processor feedback data;
providing the feedback data to the feedback module; and
the feedback module providing feedback to the user.

### BRIEF DESCRIPTION OF THE DRAWINGS

Some embodiments of apparatus and/or methods in accordance with embodiments of the present invention are now described, by way of example only, and with reference to the accompanying drawings, in which:

FIG. 1a is an external view of a behaviour capture device according to a first embodiment of the present invention;

FIG. 1b is a block diagram of the components in a behaviour capture device of FIG. 1a;

FIG. 2 is a flowchart showing a process of operation of the behaviour capture device of FIG. 1a;

FIG. 3 is a block diagram of components of a behaviour capture device according to a second embodiment of the present invention;

FIG. 4 is a block diagram of components of a behaviour capture device according to a third embodiment of the present invention;

FIG. 5 is a flowchart showing a process of operation of the behaviour capture device of FIG. 4;

FIG. 6 is schematic diagram a behaviour capture system according to a further embodiment of the invention;

FIG. 7 is a flowchart showing a process of operation of the behaviour capture system of FIG. 6;

FIG. 8 shows screenshots of a mobile phone application forming part of the system of FIG. 6;

FIG. 9 is a block diagram of a behaviour capture system according to a further embodiment of the invention;

FIG. 10 is a block diagram of a behaviour capture system according to a further embodiment of the invention; and

FIG. 11 is a flowchart showing a process of operation of the system of FIG. 10.

### DETAILED DESCRIPTION OF THE DRAWINGS

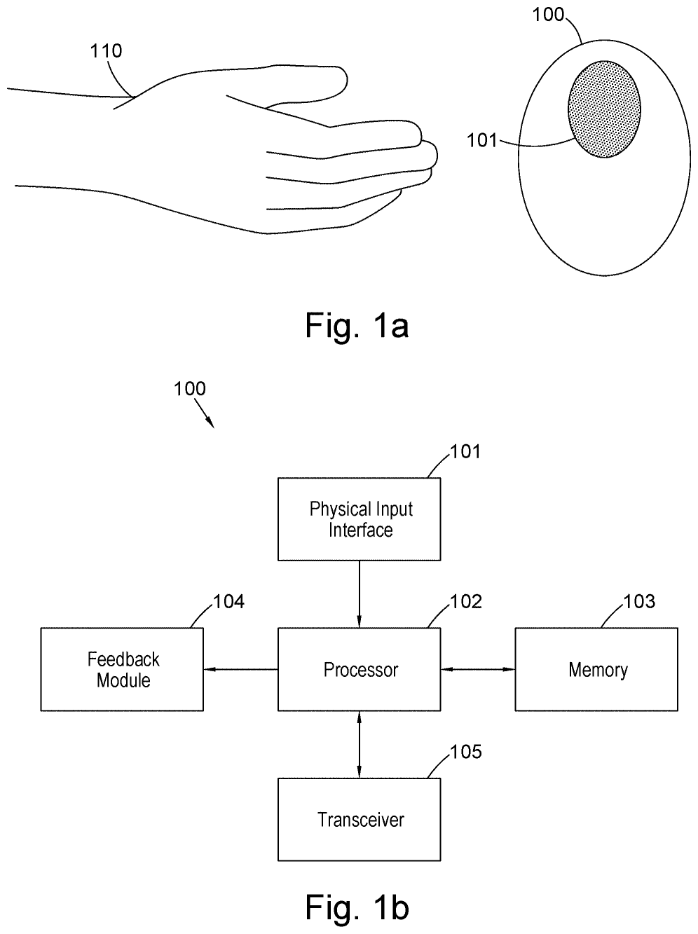

FIG. 1a shows an external view of a handheld behaviour capture device 100 in accordance with a first embodiment of the present invention. The behaviour capture device 100 is generally egg-shaped but can be any shape, size and build that allows a user to conveniently carry and/or wear, and handle, the behaviour capture device 100 in the user's hand 110. The behaviour capture device 100 comprises an electrical force or pressure sensor/push-button 101 which acts as a physical, resiliently deformable input interface for the user to provide inputs to the behaviour capture device 100.

FIG. 1b is a block diagram of the behaviour capture device 100 of FIG. 1. The behaviour capture device 100 comprises a processor 102 that is in data communication with the push button/physical input interface 101 and is arranged to receive input data from the physical input interface 101. Furthermore, the processor 102 is also arranged to be in data communication with a memory module 103, a feedback module 104, a transceiver 105 and a battery module (not shown). The processor 102 is arranged to transmit and receive data to and/or from the other components of the behaviour capture device 100, as well as process said data.

The physical input interface 101 is arranged to receive a user input. As stated above, in this embodiment the physical input interface 101 is an electrical push button. In other embodiments the physical input interface 101 can be any type of sensor which receives a user input when the user squeezes, presses or otherwise physically manipulates an effective area of the physical input interface 101 on the behaviour capture device 100. The effective area of the physical input interface 101 may be limited to a specific region of the behaviour capture device 100 such that the user must physically manipulate that specific region in order to provide a user input to the physical input interface 100. Alternatively, the effective area of the physical input interface 101 may cover the entirety of the behaviour capture device 100 such that the user can manipulate any part of the behaviour capture device 100 to provide a user input to the physical input interface 101. For example, the physical input interface 101 may comprise one or more of a pressure pad, button, or other physical input means that is capable of detecting an applied force and preferably an applied pressure

A force sensing resistor (FSR) can be used to measure the squeezing force of the user's hand. The resistance of the FSR decreases when squeezed and such a sensor can be arranged to vary a voltage received by the processor, which can be recorded as an indication of the intensity of a bookmarking behaviour, as described below, the user wishes to express. The intensity of a bookmarking behaviour may additionally be expressed by the duration that the applied pressure is above a predetermined threshold. The threshold of applied pressure may be adjustable or customisable by the user. Alternatively the integral of pressure applied over the duration of a user's squeeze can be measured as an indication of intensity of a bookmarking behaviour. If the intensity of a bookmarking behaviour is expressed solely by the duration of the squeeze, then it is unnecessary to resolve the degree of pressure being applied. A simple switch arranged to operate when pressure is applied is sufficient.

Upon receiving a user input, the physical input interface 101 is arranged to generate input data. In its most basic embodiment, where the physical input interface 101 is a push button, the input data corresponds to the closing of the electrical switch of the push button. The input data generated by the physical input interface 101 may comprise data corresponding to the force or pressure applied to the physical input interface 101 by the user. The physical input interface 101 is arranged to be in data communication with a processor 102 such that the physical input interface 101 transmits any generated input data to the processor 102. Preferably, the physical input interface 101 is arranged such that a user must apply an intentional amount of pressure to the physical input interface 101, for example that is higher than that applied during simple and regular handling of the behaviour capture device 101, in order for the physical input interface 101 to generate input data. Alternatively, the physical input interface 101 generates input data in response to all detectable pressure applied to the physical input interface 101, and the processor 102 instead provides a thresholding function to ignore input data that does not correspond to an intentional user input.

The processor 102 is configured to detect patterns of user input by analysing the input data received from the physical input interface 101. Preferably, the processor 102 is configured to detect at least two or more different patterns of user input by analysing the input data. These patterns of user input are described more generally below as “predetermined identifiers”. For example, the processor 102 may be configured to detect: a first pattern of user input by analysing the input data, wherein the first pattern of user input corresponds to one short squeeze followed by one long squeeze of the behaviour capture device 100 by a user; and a second pattern of user input by analysing the input data, wherein the second pattern of user input corresponds to two short squeezes followed by one long squeeze of the behaviour capture device 100 by a user. It should be noted that different recognisable patterns of user input detectable by the processor 102 are also within the scope of the present invention.

Additionally, the processor 102 is configured to analyse input data received from the physical input interface 101 and determine secondary data from the input data. For example, the processor 102 may determine secondary data from input data wherein the secondary data corresponds to at least one of: the length of time of each user input; the amount of pressure applied during user input; and the frequency of user input. The processor 102 is also configured to determine the current time and date using a clock (not shown).

The memory module 103 is arranged to receive, store and transmit data to and from the processor 102. The data received by the memory module 103 includes input data transmitted from the physical input interface 101, via the processor 102. The memory module 103 comprises a database for storing data records of data received from the processor 102. The memory module 104 is also arranged to transmit the data stored in the database to the processor 102.

The feedback module 104 is arranged to provide user feedback in response to usage of the behaviour capture device 100, based on instructions received from the processor 102. Preferably, the feedback module 104 comprises a haptic feedback motor. The instructions received from the processor 102 include instructions to activate and deactivate the feedback module 104. Preferably, the feedback module 104 is able to exhibit different patterns of feedback depending on the instructions received from the processor 102. For example, the haptic feedback motor in the feedback module 104 is preferably arranged to pulse or remain on for different duty cycles and periods of times based on the instructions received from the processor 102. Preferably, the feedback module 104 provides feedback in response to each user input. For example, for each short squeeze of the behaviour capture device 100, the haptic feedback motor in the feedback module 104 activates correspondingly with a short activation. The feedback module 104 is also arranged to provide notifications to the user through, for example, the activation of the haptic feedback motor.

In an alternative embodiment, the feedback module 104 also includes display or illumination means, such as an LED array. Preferably, the LED array is arranged to exhibit different patterns of feedback based on instructions received from the processor 102. For example, the LEDs in the LED array may turn on or off individually or in groups depending on the instructions received from the processor 102. Additionally, in this embodiment, the LED array is arranged to display system information concerning the behaviour capture device 100. This may include using at least one of the LEDs in the LED array to indicate: a battery level of the behaviour capture device's 100 battery module; and/or the state of each operating mode of the behaviour capture device 100. The operating modes of the behaviour capture device 100 may be one or more of: airplane mode, sleep mode, on/off mode and battery mode.

Turning back to the first embodiment in FIG. 1b, the transceiver module 105 is arranged to send and receive data, to and from the processor 101. The transceiver module 105 is also arranged to send and receive data to and from an external transceiver. Preferably, the external transceiver is part of, for example, an external mobile device. The transceiver module 105 is arranged to pair or connect with the external transceiver via Bluetooth (®) Low Energy (BLE), Wi-Fi (®), cellular network or through any other means of wireless communications. Additionally, the transceiver module 105 is configured to allow communication between the behaviour capture device 100 and a server.

Although not shown in FIG. 1, the battery module of the behaviour capture device 100 is arranged to provide power to the components of the behaviour capture device 100. Preferably, the battery or batteries in the battery module is one of a lithium ion battery, lithium polymer battery, nickel metal hydride battery, or any other battery suitable for the behaviour capture device 100 or other Internet of Things (IoT) devices. The battery module is also arranged to charge the battery using one or more wired or wireless charging methods.

The behaviour capture device 100 can be used by a user to actively record moods, thoughts, feelings, emotions, actions, habits, behaviours or other information related to the user's psychological and/or physical state or behavioural health. In the description that is to follow, these are simply referred to as “behaviours”, although they are not limited as such. Using, for example, the companion mobile application to the behaviour capture device 100, the user can pre-assign behaviours to be tracked that are relevant to their goals and objectives. A user can then use the behaviour capture device 100 to record each time the user experiences such pre-assigned behaviours. The recordal of these pre-assigned behaviours is herein referred to as creating a ‘bookmark’. The pre-assigned behaviours identified by a user for bookmarking may be changed by the user at their discretion. The number of pre-assigned behaviours, referred to as ‘bookmark types’, available for recordal corresponds to the number of patterns of user input or predetermined identifiers the processor 102 of the behaviour capture device 100 is capable of detecting. For example, if the processor 102 is configured to detect two predetermined identifiers corresponding to two respective user input patterns, then the user has two bookmark types at their disposal for recordal, i.e., type 1 and type 2. If the two detectable input identifiers are one short squeeze followed by one long squeeze of the behaviour capture device 100; and two short squeezes followed by one long squeeze of the behaviour capture device 100, then these two user input identifiers/patterns correspond to bookmarks type 1 and type 2, respectively. In general each bookmark type corresponds to a predetermined identifier or pattern of user inputs. In particular, each bookmark type corresponds to a plurality of distinct user inputs of predetermined duration.

Prior to using the behaviour capture device 100, the user will have associated a behaviour with each bookmark type, which forms the predetermined set of behaviours. For example, a user may pre-assign a behaviour with each bookmark type in the course of initially configuring the behaviour capture device 100 using an external program or other device. Hence, in order to record a specific behaviour from the predetermined set of behaviours, the user will record the corresponding bookmark type by inputting the corresponding user input pattern, herein referred to as performing a ‘bookmarking action’.

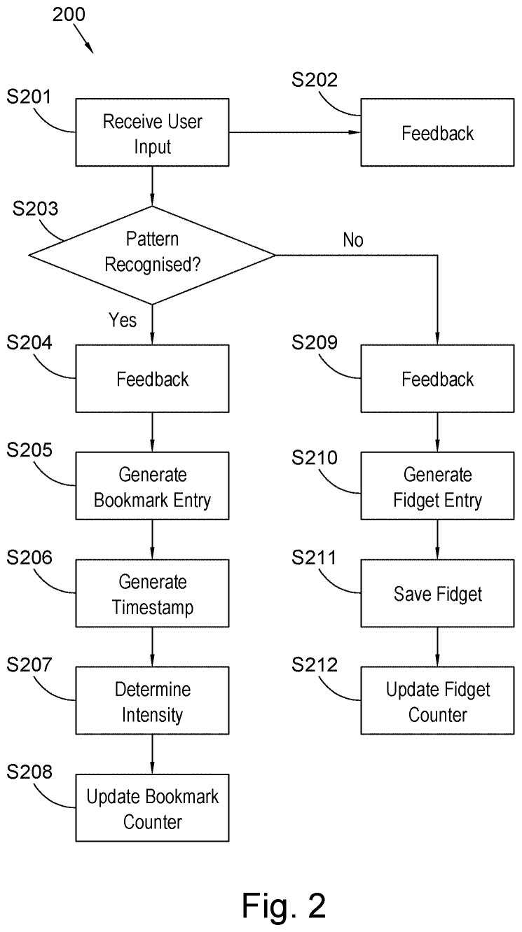

FIG. 2 is a flowchart showing how the behaviour capture device 100 can be used to record a bookmark input or event. At step S201, the behaviour capture device 100 receives a pattern of user inputs to initiate a bookmark of the corresponding type, wherein the initiated bookmark type corresponds to the behaviour that the user wishes to record. Providing user input to the behaviour capture device 100 involves exerting pressure on or squeezing at least part of the behaviour capture device 100 that is detectable by the physical input interface 101. The physical input interface 101 detects the user input and generates corresponding input data. Each user input data generated by the physical input interface 101 in response to receiving a user input is transmitted to the processor 102.

At step S202, in response to each user input, the processor 101 instructs the feedback module 104 to activate and provide feedback. Preferably, the instructions from the processor 102 are dependent on the user input data received by the processor 101 from the physical input interface 101, and therefore the feedback module 104 exhibits different feedback patterns dependent on the user input data received by the processor 102. For example, a haptic feedback motor in the feedback module 104 may activate in short bursts during short squeezes of the behaviour capture device 100, and provide a pulsing sensation during longer squeezes of the behaviour capture device 100. Similarly, an LED array in the feedback module 104 may activate in short bursts during short presses of the behaviour capture device 100, and provide a pulsing glow during longer squeezes of the behaviour capture device 100. Additionally, the haptic feedback motor may provide more powerful feedback corresponding to the amount of pressure exerted on the behaviour capture device 100 by the user, or the LED array may light up brighter corresponding to the amount of pressure exerted on the behaviour capture device 100 by the user. Additionally, the number of activated LEDs in the LED array may correspond to the amount of pressure exerted on the behaviour capture device 100 by the user such that a higher pressure or tighter squeeze corresponds to more activated LEDs. It should be noted that different patterns of feedback in response to user input are within the scope of the invention.

At step S203, the processor 102 determines whether the user has provided a recognisable pattern of user input, i.e., performed a bookmarking action, by analysing the input data received from the physical input interface 101. If a bookmarking action is detected, at step S204, the feedback module 104 provides a pattern of feedback that confirms to the user that a bookmark action has been detected. The pattern of feedback provided by the feedback module 104 when confirming the detection of a bookmark action to the user may be any predetermined pattern of haptic and/or light feedback. At step S205, the processor 102 instructs the memory module 103 to create a new bookmark record in the database that corresponds to the bookmark type.

At step S206, the processor 102 associates a time stamp with the new bookmark and stores it in the database in the memory module 103 in association with the new bookmark entry or record. The time stamp is indicative of the time and date on which the new bookmark was created.

At step S207, the processor 102 determines and associates an intensity with the new bookmark and stores it in the database in the memory module 103 in association with the new bookmark record. The intensity is indicative of the user's evaluation of the strength of the corresponding behaviour. The processor 102 analyses the input data received from the physical input interface 101 to determine the intensity. Preferably, the processor 102 determines the intensity based on the length of the long squeeze stage of the user input patterns described above, wherein a longer squeeze indicates a higher intensity. Alternatively, the processor 102 may determine the intensity based on other factors such as: the total amount of pressure applied during the bookmaking action; or the total length of time of the bookmarking action. In other embodiments this step may be skipped and no intensity data may be determined or recorded.

At step S208, the processor 102 updates a record in the database in the memory module 104 indicating the number of times that that bookmark type has been recorded.

Thus, at the end of the bookmarking process shown in FIG. 2, the behaviour capture device 100 stores a bookmark corresponding to the pre-assigned behaviour that the user was feeling, as well as the time, date and, in some cases, the intensity of the bookmarking event. The bookmarking action described above allows a user of the behaviour capture device 100 to easily and intuitively record each time the user experiences a behaviour that they are tracking for the purpose of, for example, assessment, behaviour management, adaptation, mindfulness, adjustment, and/or behaviour change.

FIG. 2 also shows the steps taken where the user 110 provides a user input to the behaviour capture device 100 that is not in accordance with a pattern corresponding to a bookmark type, herein referred to as a ‘fidget’ action or a non-bookmark input or event. Where bookmarks are for recording specific behaviours, a fidget or non-bookmark input instead represents general usage and engagement with the behaviour capture device 100. Hence, at step S203, if the processor 102 determines that the user has not provided a pattern of user input corresponding to a bookmark type, the processor 101 determines that the user has made a fidget action as opposed to a bookmarking action. At step S209, the feedback module 104 provides a pattern of feedback that confirms to the user that a fidget or non-bookmark event has been detected. The pattern of feedback provided by the feedback module 104 may be any pattern of haptic and/or light feedback that allows a user to differentiate between the detection of a bookmark action and non-bookmark event. In an alternative embodiment, the absence of any feedback may suffice for this effect. At step S210, the processor 102 instructs the memory module 103 to create a new fidget record in the database.

At step S211, the processor 102 stores the fidget to the memory module 103. Storing the fidget to the memory module 103 may include storing any captured pressure data as a function of time.

At step S212, the processor 102 updates a record in the database in the memory module 104 indicating the number of times that a fidget has been recorded as a non-bookmark record.

Thus, at the end of the fidget process shown in FIG. 2, the behaviour capture device 100 stores recorded data in association with a fidget, in some cases including pressure data as a function of time.

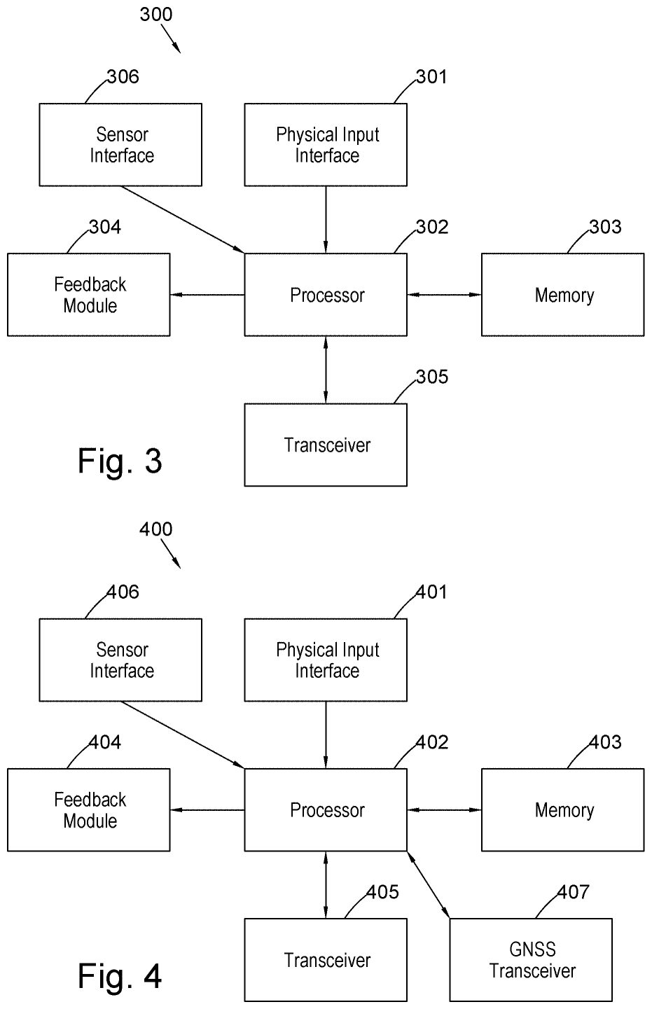

FIG. 3 shows a second embodiment of the present invention. The behaviour capture device 300 has the same features as the behaviour capture device 100 shown in FIGS. 1a and 1b with additional features as described below. The behaviour capture device 300 further comprises a sensor module 306. The sensor module 306 is in data communication with the processor 302 and is arranged to provide sensing data to the processor 302. The sensor module 306 comprises one or more of an accelerometer, magnetometer, or any other sensor that is capable of producing sensing data that is indicative of the movement of the behaviour capture device 300. Preferably, the sensing data includes data indicative of the orientation of the behaviour capture device 300, and data indicative of the acceleration forces applied to the behaviour capture device 300. Preferably, the sensor module 306 continuously provides sensing data to the processor 302 whilst the behaviour capture device 300 is powered on. Preferably, the processor 302 continuously stores the sensing data received from the sensor module 306 to the memory module 303. Alternatively, the processor 302 only stores sensing data to the memory module 303 when the sensing data is above a certain threshold. I.e., the processor 302 only stores sensing data to the memory module 303 when the behaviour capture device 300 is detected to be sufficiently active or moving, in order to avoid the storage of unnecessary sensing data.

Storing data indicative of the movement of the behaviour capture device 300 using the sensor module 306 allows for monitoring of the user's passive engagement with the behaviour capture device 300. An advantage of this is that such data can be used in combination with bookmark and fidget data to infer useful information about the user's usage of the behaviour capture device 300. For example, if there are long periods of time where the user has not actively recorded any bookmarks or fidgets, the sensor data may be used to determine whether the user has been moving or handling the device 300 in other ways. If so, it can be inferred that the user is using the device 300 but has not felt the need to record bookmarks and fidgets, as opposed to simply neglecting the device 300.

In an alternative embodiment, the sensor module 306 further comprises physiological sensors, which may include Galvanic Skin Response (GSR) sensors and pulse or heart rate sensors. Preferably, the physiological sensors also continuously provide sensor data to the processor 302. Preferably, the processor 302 stores the sensor data in the memory module 303 when the processor 302 detects that the physiological sensors are in use. When the processor 302 detects that the physiological sensors are in use, the processor 302 stores a GSR, pulse or heart rate reading in the memory module 303 with an associated time stamp.

The collection of physiological data of the user in the above alternative embodiment provides additional data to complement bookmark, fidget and movement sensor data. Particularly, physiological data can be used in combination with the bookmark and fidget data to infer useful information and verify trends. For example, patterns of stress indicated by GSR readings and pulse or heartbeat readings can be compared at the times a user records a bookmark associated with stress. Hence, such comparisons may allow the verification of certain behaviour capture and data patterns.

FIG. 4 shows a third embodiment of the present invention. The behaviour capture device 400 has all the features of the first and second embodiments, with additional features as described below. The behaviour capture device 400 of the third embodiment differs in that it has a GNSS (Global Navigation Satellite System) receiver 407. The GNSS receiver 407 is in data communication with the processor 402 and is arranged to provide location data to the processor 402 when requested by the processor 402. Preferably, location data provided by the GNSS receiver 407 comprises coordinate data, and time stamps associated with the coordinate data indicating the time and date on which the coordinate data was received by the GNSS receiver 407. Hence, according to the third embodiment of the behaviour capture device 400, the processor 402 has the additional functionality of being able to request and receive location data from the GNSS receiver 407.

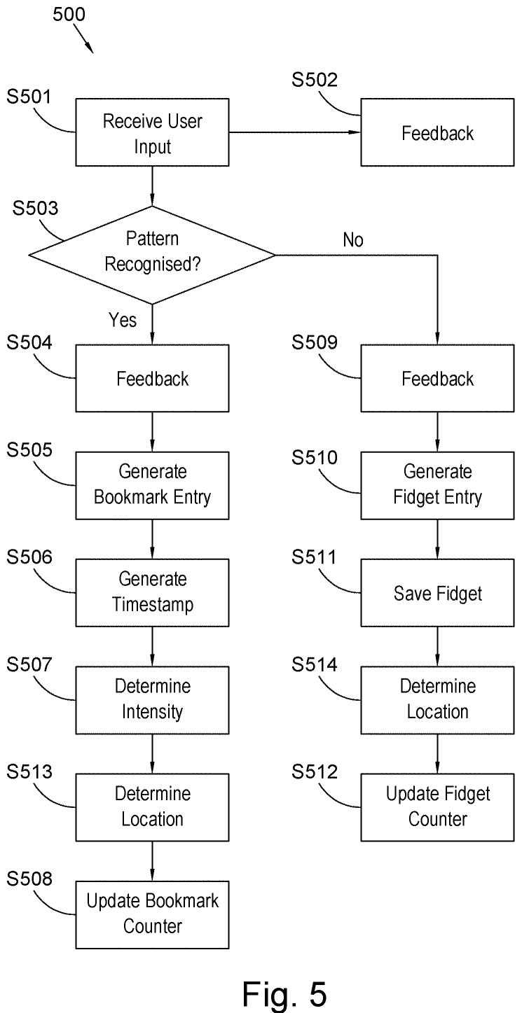

The purpose of the GNSS receiver 407 is to record additional location data when a user records a bookmark or a fidget. An example of this is shown in the flow chart in FIG. 5, which shows the process for recording a bookmark and fidget using the behaviour capture device 400. The steps in FIG. 5 are mostly similar to those in FIG. 2. However, the process for recording a bookmark in FIG. 5 includes the additional steps S513 and S514. At step S513, the processor 402 sends a request to the GNSS receiver 407 to be provided with the location of the behaviour capture device 400. Once the GNSS receiver 407 has received the location data, the GNSS receiver 407 transmits the location data to the processor 402. The processor 402 then stores the location data in the database in the memory module 403 in association with the new bookmark record.

Similarly, at step S514, the processor 402 sends a request to the GNSS receiver 407 to be provided with the location of the behaviour capture device 400. Once the GNSS receiver 407 has received the location data, the GNSS receiver 407 transmits the location data to the processor 402. The processor 402 then stores the location data in the database in the memory module 403 in association with the new fidget entry or record.

The GNSS receiver 407 and its functionality provides contextual information to complement bookmark and fidget data. The location data may be used to infer further insights in association with the recorded bookmarks and fidgets. For example, one may use the location data to associate links between a user's location and their behaviour recordal activities. Additionally, in a case that the user later inspects their recorded bookmarks and fidgets, having associated location data may be advantageous so that the user can more easily remember contextual information surrounding the behaviour recordal, such as the environment, who they were with, what they were doing, and why they felt that behaviour.

In an alternative embodiment, the processor 402 is arranged to continuously receive location data from the GNSS receiver 407 whilst the behaviour capture device 400 is powered on. Furthermore, the processor 402 is arranged to store all received location data to the memory module 403. Alternatively, the processor 402 only stores location data to the memory module 404 when the location changes by a significant amount, by a distance exceeding a predetermined threshold. This functionality of the present embodiment allows for the continuous tracking of the behaviour capture device 400 whilst it is powered on. Storing a continuous stream of location data would allow one to analyse a user's general engagement with the behaviour capture device 400. The continuous location data may also allow one to verify a user's usage of the behaviour capture device 400, during periods of fewer bookmark and fidget recordals. For example, it is expected that the user will interact less frequently with the behaviour capture device 400 whilst they are driving.

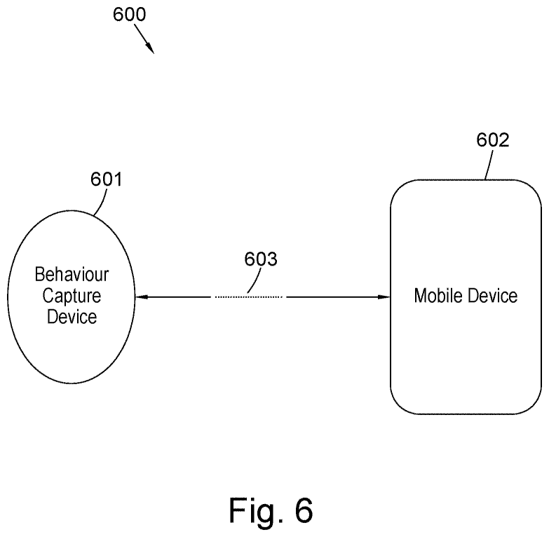

FIG. 6 shows a first arrangement of a behaviour capture system 600 in accordance with a further embodiment of the invention. The behaviour capture system 600 comprises a behaviour capture device 601 in accordance with one of the embodiments of the invention described above. The behaviour capture device 601 is arranged to be in data communication with a mobile device 602 over a wireless network 603. The mobile device 602 may be a mobile phone, a tablet device, a notebook, a laptop or other similar portable computing device. Preferably, the behaviour capture device 601 and mobile device 602 are only in data communication after the completion of a pairing procedure. Preferably, the behaviour capture device 601 is arranged to pair to the mobile device 602 using Bluetooth (®) Low Energy, WiFi (®), cellular network or any other suitable form of wireless communication protocol. The behaviour capture device 601 is arranged such that, when paired or bonded to the mobile device 602, the behaviour capture device 601 is able to transmit and receive data to and from the mobile device 602. Another mechanism for enabling a one-to-one relation for identification and communication to be established may be used, and is referred to herein as “pairing”. Particularly, when paired to the mobile device 602, the behaviour capture device 601 is able to send all data that is stored in the memory module 103, 303, 403, including bookmark data, fidget data, sensor data, location data and all associated data including time-stamps, locations and intensities. Collectively, this is herein referred to as behaviour capture data. The behaviour capture device 601 is also arranged to send system data to the mobile device 602, including the battery level and state of operating modes of the behaviour capture device 601. Furthermore, when paired to the mobile device 602, the behaviour capture device 601 is able to receive data including: system messages; instructions to provide feedback; and instructions to change the operating mode of the behaviour capture device 601.

The mobile device 602 is arranged to allow data communication between the behaviour capture device 601 and the mobile device 602 after the completion of a pairing procedure. The mobile device 602 is configured to input, transmit, receive, process and display information. Furthermore, the mobile device 602 is host to a mobile application that allows the user to interact with the behaviour capture device 401 and behaviour capture data. The mobile device 602 also stores the behaviours that are associated with the available bookmark types.

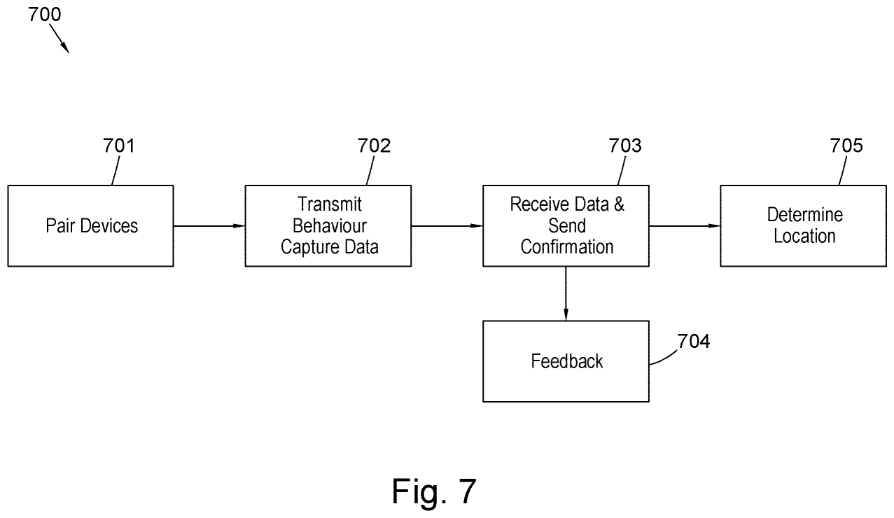

A user can use the mobile device 602 to retrieve and store the behaviour capture data that has been collected using the behaviour capture device 601, the process for which is shown in FIG. 7. At step 701, the mobile device 602 is first paired or bonded to the behaviour capture device 601 to enable data communication between the mobile device 602 and the transceiver 105, 305, 405 of the behaviour capture device 601.

Once paired, in step 702, the behaviour capture device 601 transmits all new behaviour capture data to the mobile device 602. This includes the transmission of bookmark and fidget records in the database of the memory module 103, 303, 403 and their respective types, time-stamps and intensities. The transmitted data may also include the sensor data stored in the memory module 303, 403 collected using the sensor module 306, 406. Additionally, the transmitted data may also include location data in accordance with the third embodiment of the invention.

At step 703, the mobile device 602 receives the transmitted behaviour capture data. Upon receiving the transmitted behaviour capture data, the mobile device 602 associates a corresponding behaviour with each received bookmark based on predetermined bookmarks types (e.g., bookmark types 1 and 2). Furthermore, upon successfully receiving the information, the mobile device 602 sends a confirmation message to the behaviour capture device 601.

At step 704, the behaviour capture device 601 receives the confirmation message. Upon receiving the confirmation message, the processor 102 of the behaviour capture device 601 instructs the feedback module 104, 304, 404 to provide user feedback. Preferably, the feedback pattern exhibited by the feedback module 104, 304, 404 is a pattern that notifies the user. For example, a haptic feedback motor in the feedback module 104, 304, 404 may activate and exhibit a pattern of feedback that notifies the user of the confirmation. Alternatively, one or more LEDs in an LED array in the feedback module 104, 304, 404 may blink or light up to notify the user of the confirmation. It should be noted that feedback from the LEDs and haptic feedback motor may be exhibited at the same time. Furthermore, preferably, the behaviour capture device 601 automatically deletes all transmitted data from its own memory module 103, 303, 403 upon receiving the confirmation message from the mobile device 602, in order to make memory space available for subsequent behaviour, location and sensor data capture. Alternatively, the behaviour capture device 601 does not automatically delete the transmitted data in response to the confirmation message, and only deletes the data when instructed to by the mobile device 602 in a separate instruction. For example, the mobile application on the mobile device 602 may have a specific function to send an instruction to delete old data from the behaviour capture device 601. Alternatively, the behaviour capture device 601 may comprise a button or switch to delete old behaviour capture data.

At step 705, once the mobile device has stored the received behaviour capture data, the mobile device 602 associates the current location (where available) of the mobile device 602 with the received data using the mobile device's GNSS capabilities. However, the skilled person will appreciate that this step can be omitted if the behaviour capture device 601 is in accordance with the third embodiment of the invention shown in FIG. 4.

After the operation in FIG. 7, the user may keep the behaviour capture device 601 paired to the mobile device 602. In this case, any newly recorded bookmarks and fidgets, or new sensor data, will be immediately uploaded to the mobile device 602. This may be advantageous if the user wishes to immediately access their behaviour capture data. Furthermore, if the behaviour capture device 601 in accordance with the first and second embodiments is kept paired to the mobile device 602, the GNSS location associated with each recorded bookmark and fidget will more accurately represent the location of the user when the bookmark or fidget was created, as opposed to when the data was uploaded to the mobile device 602. This is advantageous since it would allow one to infer trends and patterns by linking location data to bookmark data, as well as to gauge the user's usage of the behaviour capture device 601, the advantages of which were discussed above.

Alternatively, the user may disconnect the behaviour capture device 601 from the mobile device 602. In this case, the user may continue to record bookmarks, fidgets and other data on the behaviour capture device 601, and transfer the data at a later time to the mobile device 602 as described in FIG. 7. This may be advantageous if the user wishes to preserve battery life on both the behaviour capture device 601 and the mobile device 602, since maintained pairing may use more power on both devices. Furthermore, this operation may be advantageous if the user's mobile device 602 is not in close proximity to the behaviour capture device 601, since the wireless pairing may have proximity limitations.

Storing behaviour data on the mobile device 602 has the advantage of making the data more accessible through the mobile device's extended network capabilities and more accessible user interface. Furthermore, storing the behaviour data on the mobile device 602 provides the user with a backup of the data as a security measure, where the data can be further replicated and backed up by transferring it from the mobile device 602 to a server or other computer storage media. Additionally, the mobile device 602 may have more processing power than the behaviour capture device 601 and therefore allow for more intensive processing of the behaviour capture data.

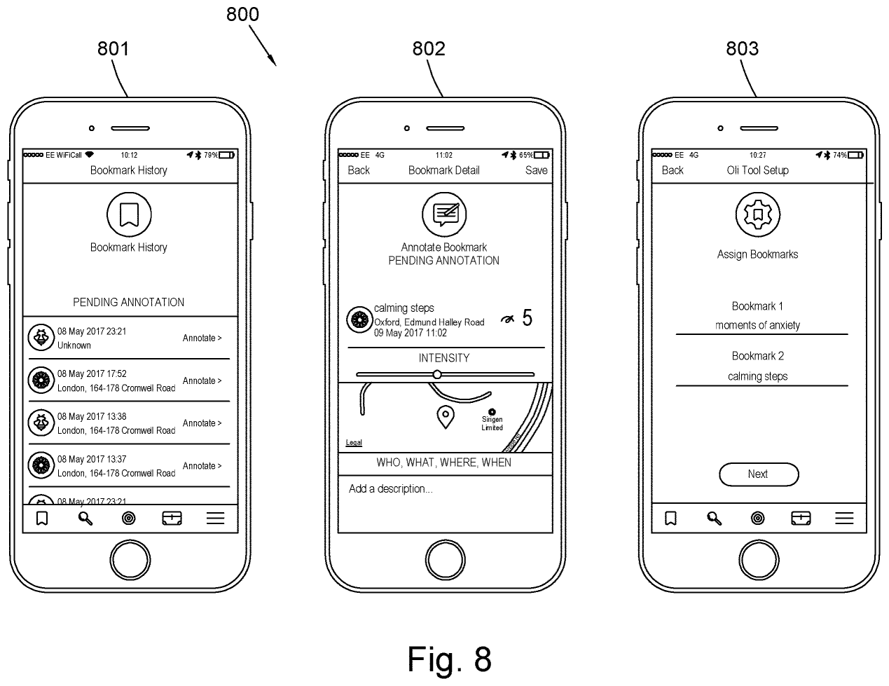

A user can also use the mobile device 602 to view, annotate and analyse the behaviour capture data using a mobile application that is hosted on the mobile device 602, providing that such data has been transferred to the mobile device 602 through the process in FIG. 7 or otherwise. FIG. 8 shows examples of different screenshots of the mobile application. View 801 shows a history of recorded bookmarks. The behaviour that was associated with the bookmark type at the time of recordal is listed, along with the time and date on which the bookmark was created. Preferably any new bookmarks received by the mobile device 602 are appended or merged with the existing bookmarks on the mobile device 602. From this view 801, the user has the option to view each bookmark in more detail and provide annotations of contextual information. Bookmarks that are yet to be annotated are grouped or flagged. The user also has the option to delete bookmarks.

View 802 shows how a bookmark can be annotated with contextual information. The view 802 displays the behaviour related to the bookmark, the address and map location associated with the bookmark, and an option to edit the intensity of the bookmark. Although the intensity of a bookmark is determined at the time of recordal using the behaviour capture device 601, the option to edit the intensity is provided for the user to edit it in retrospect. The user is able to annotate the bookmark with text. Annotations provided by the user preferably include contextual information concerning: where the user was, what the user was doing and who the user was with at the time of recording the bookmark; as well as why the user believes he felt the need to record the bookmark. Annotations may also include any other information that the user feels is relevant to the context of the bookmark. Alternatively, annotations may also be provided by the user in the form of images and ‘stickers’, or a selection of images, phrases and number ratings from a limited range of options. Annotations may also include audio clips uploaded to the behaviour capture device or the mobile device. By annotating bookmarks with contextual information, the user is able to reflect upon and determine the reasons or causes surrounding their behaviour. Hence, the user can use this as feedback to work towards their behaviour change targets. The mobile application may also include avatars to assist in the behavioural understanding and learning process for children, which in turn may be linked to the sending of messages or haptic feedback.

View 803 shows a page where a user can change the behaviour that is associated with a bookmark type. For example, the user can change the behaviour that is associated with bookmark type 1 if s/he decides to track a different behaviour using the behaviour capture device 601. After assigning a new behaviour to bookmark type 1 using the mobile device 602, every new bookmark of type 1 that is received by the mobile device 602 and was recorded on the behaviour capture device 601 after the new behaviour assignment will be stored in association with the new behaviour. All previous bookmarks of type 1 that were recorded before the new behaviour assignment will remain in association with the behaviour that was previously associated with bookmark type 1.

The ability to customise the behaviour associated with bookmark types provides the user with flexibility as to how they use the behaviour capture system 600 for their own personal targets. For example, a behaviour that a user chooses to associate with a bookmark type may be a particular behaviour, thought, feeling, action, or other information related to the user's psychological and physical state. In one example, a user may use the behaviour capture system 600 to track and manage stress. This could be achieved by associating feelings of stress with bookmark type 1. The user may track feelings of stress using bookmark type 1 alone, or use bookmark type 2 in combination with bookmark type 1 depending on the user's stress management targets. For example, the user may associate events of stress externalisation with bookmark type 2 to track when the user acts upon his feelings of stress. Alternatively, the user may associate instances of positive stress management with bookmark type 2, to track when the user successfully manages and overcomes feelings of stress in a positive way. In either case, the user is able to track feelings of stress, the context surrounding these feelings through annotations, and other information such as location and sensor data to work towards his/her behaviour change target.

In another example, a user may use the behaviour capture system 600 to track and manage a treatment plan for pain management, including physiotherapy and cognitive therapy. A user may associate feelings of pain with bookmark type 1, and the action of doing cognitive therapy homework with bookmark type 2. Alternatively the user may associate instances of positive pain management with bookmark type 2, such as breathing exercises or other predetermined activities. Such tracking can help the user to analyse the effectiveness of their treatment plan for pain management or other actions. Such data can also be used by professionals to assist in pain management to achieve maximum effectiveness and efficiency by considering patterns in the tracking data.

In another example, a child may use the behaviour capture system 600 for social and emotional learning to help them better understand how their behaviours are connected to their emotions. A child user may associate feelings of anger with bookmark 1, and the action of doing cognitive homework to counter the anger with bookmark 2. Such tracking can help the child user to learn about their feelings of anger and associated behaviours and how to manage those feelings and change their challenging behaviour.

In another example, a child may use the behaviour capture system 600 to help them manage ADHD or help adults in detecting behavioural challenges. A child user may associate bookmark type 1 with feeling distracted, needing to move around or take a break from concentrating. A child user may associate a bookmark type 2 with actions of doing homework to manage their attention to stay on task. Such tracking can help the child user and supervising adults to analyse the situations and patterns that precipitate the inattentive behaviour or other ADHD symptoms and plan a strategy for managing these. Such data can also be used by the adults to determine the effectiveness of any behaviour strategies which have been implemented.

In another example, a user may use the behaviour capture system 600 to track and manage smoking cessation. A user may first associate the feeling of cravings with bookmark type 1 to assess patterns in the user's cravings, and associate bookmark type 2 with stress management techniques, such as exercise. Alternatively the user may associated bookmark type 2 with cigarette smoking. The user may use this data to alter their daily routine and/or adjust nicotine substitutes to achieve maximum benefit and efficiency for tackling smoking addiction. It should be appreciated that such use of the behaviour capture system 600 may be extended to any other form of addiction and withdrawal symptoms. In any case, the behaviour capture system 600 allows the user to work towards their behaviour change target.

Furthermore, if there are at least two bookmark types available, i.e., types 1 and 2, the user may associate behaviours in an opposite way. For example, the user may associate feelings of anxiety with type 1 and confidence with type 2 in order to track these opposite psychological states. In this case, the user may spot patterns in these states by considering their times, locations, and contextual annotations surrounding it. Alternatively, the user may associate behaviours in an additive way. For example, the user may associate feelings of anxiety with type 1, and feelings of deeper anxiety with type 2. In this case, the user is able to more easily separate different causes of the behaviours and their effect at a more separate or granular scale.

The mobile application on the mobile device 602 is able to process all behaviour capture data and annotations to perform analytics and machine learning. This may include, for example, scanning bookmark annotations for keyword analysis; and analysing bookmark, fidget, location and sensor data to recognise patterns and trends in behaviour and usage of the behaviour capture device 601. Hence, based on the performed analytics and machine learning, the mobile application is able to provide the user with notifications, warnings, customised goals, wellbeing messages or any other personalised user content.

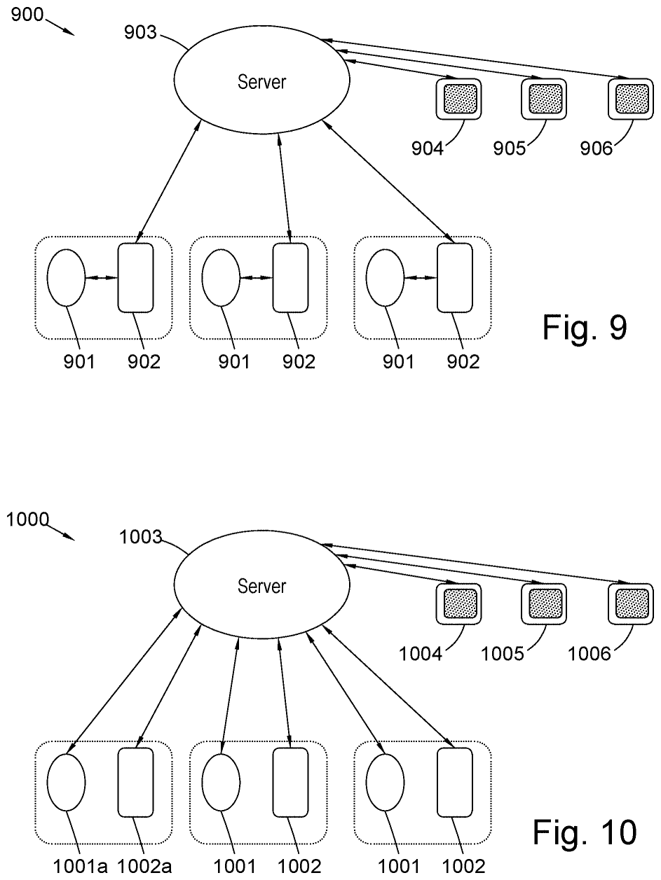

Furthermore, the mobile application is able to display bookmark, fidget, location, sensor and usage data in various graphical forms. It is also able to perform other functions such as sending messages and uploading files. Additionally, whilst the mobile device 602 is paired or bonded to the mobile device 601, the mobile application is able to: display the battery level of the behaviour capture device 601; and view and change the operating modes of the behaviour capture device 601. This includes, for example, the enabling and disabling of airplane mode, sleep mode, on/off mode and battery mode. FIG. 9 shows a second arrangement of a behaviour capture system 900 in accordance with a further embodiment of the invention. The behaviour capture system 900 comprises a plurality of behaviour capture devices 901, where each behaviour capture device 901 is in accordance with the first, second or third embodiments of the invention. Each behaviour capture device 901 is arranged to be in data communication with a mobile device 902. Hence, each pair of behaviour capture device 901 and mobile device 902 forms a subsystem, which is in accordance with the first arrangement of the behaviour capture system 600 described in FIG. 6.

The behaviour capture system 900 includes a server 903. The server 903 is arranged to be in data communication with the mobile device 902 of each subsystem. Preferably, the server 903 and mobile devices 902 communicate over a wireless network, such as Wi-Fi (®), cellular network, or any other suitable means of wireless communication. The server 903 is arranged to store and process data received from the mobile devices 902. Data received by the server 903 includes all bookmark data, fidget data, sensor data, location data, and all associated data such as annotations, locations, time-stamps and intensities. The server 903 is also arranged to receive and store a complete backup of personal user information, behaviour change targets, behaviour assignments to bookmark types, personalised content and other data from the mobile application on each mobile device 902. Preferably, the server 903 receives data from the mobile devices 902 instantaneously, provided that there is a working connection between the server 903 and mobile devices 902.

In an alternative embodiment, the server 903 is configured to perform machine learning and analytics on the data received from the mobile devices 902, rather than the mobile devices 902 as previously described. This includes: scanning bookmark annotations for keyword analysis; and analysing bookmark, fidget, location and sensor data to recognise patterns and trends in behaviour and usage of the behaviour capture devices 902. Hence, based on the performed analytics and machine learning, the server 903 is able to transmit notifications, warnings, customised goals, wellbeing messages or any other personalised content to the user via the mobile device 902. Performing machine learning and analytics on the server 903 may be advantageous if the server 903 has a larger capacity for processing, at least relative to the mobile devices 902. Hence, performing machine learning and analytics on the server 903 may be a more efficient delegation of processing load, and consequently may help reduce processing load on the mobile devices 902. This may result in longer battery life and general longevity of the mobile devices 902 and their components.

In another alternative embodiment, the server 903 is arranged to be in data communication with external service providers. Preferably, the server 903 receives information from an external service provider over a wireless network. Preferably, the external service providers are service providers that the user receives services from, wherein the services relate to the gathering of physical or psychological data with respect to the user. The server 903 can therefore use information from a user's external service provider account in combination with a user's behaviour capture data when providing insights and personalised content through analytics and machine learning as described above.

The behaviour capture system 900 comprises at least one user terminal 904. The user terminal 904 is arranged to be in data communication with the server 903. Preferably, the user terminal 904 is in communication with the server 903 over a wireless network. Alternatively, the user terminal 904 may be in communication with the server 903 over a wired network. A user can use the user terminal 904 to access their personal behaviour capture data from the server 903. Preferably, the user accesses their personal behaviour capture data by logging into a web application or a software application. The user may log in to the user terminal 904 and access all of their behaviour capture data in graphical form or otherwise. Additionally, the user may access other personalised user content such as notifications, behaviour change targets and warnings. The user may use the user terminal 904 to perform some functions performed also by the mobile device 902, such as providing annotations to bookmarks, editing bookmarks and changing personal settings. The user terminal 904 therefore provides an alternative way for a user to access and interact with their behaviour capture data. This may be advantageous in certain circumstances, for example if the user's mobile device 902 is misplaced, malfunctioning, has run out of battery or if the user does not have a mobile device 902. Otherwise, the user terminal 904 also allows a user to access their behaviour capture data on a device that has more processing power for visual graphics and rendering, a more convenient keyboard, and more storage capacity for creating a local backup of their behaviour capture data.

The behaviour capture system 900 may also comprise at least one advocate terminal 905. In this context the advocate may be a therapist, counsellor, carer, family member etc. The advocate terminal 905 is arranged to be in data communication with the server 903 over a wired or wireless network. The advocate terminal 905 may be used by a behaviour change professional, or therapist, to access, upon authorisation, an individual's personal behaviour capture data. Preferably, the advocate's access to users' personal behaviour capture data is overseen by a system administrator, such that the advocate only has access to the data of their specific authorizing users. The advocate may use the advocate terminal 905 to visualise all of a user's behaviour capture data, such as bookmark, fidget, sensor and location data as well as all associated time-stamps, intensities and locations in graphical or raw data format. Alternatively, the advocate's access may be limited by an administrator of the behaviour capture system 900 to only access certain parts of a user's personal behaviour capture information. For example, the advocate may not be able to see a certain user's location data if not authorised to be an administrator. The advocate may use the advocate terminal 905 to provide professional comments and feedback to users of the behaviour capture system 900. Hence, the advocate may oversee and aid in a user's progression through their behaviour change programs, and develop further insights to better help a user meet their behaviour change targets.

The behaviour capture system 900 also comprises at least one administrator terminal 906. The administrator terminal 906 is arranged to be in data communication with the server 903 over a wired or wireless network. A system administrator may use the administrator terminal 906 to perform administrative tasks within the behaviour capture system 900. For example, an administrator may use the administrator terminal 906 to: manage user licences and subscriptions to the system; deploy system-wide updates; manage security of the system; grant or limit access to certain features or data to users and advocates, such as ensuring a user of a user terminal 904 may only access their own personal data and ensuring an advocate may only access their authorising users' data; send specific user or system wide messages; and generally provide system oversight and management.

FIG. 10 shows a third arrangement of a behaviour capture system 1000 in accordance with an embodiment of the invention. The behaviour capture system 1000 comprises most of the features of the behaviour capture system 900 and individual components in FIG. 9, with the main differences as described below. The behaviour capture system 1000 comprises a plurality of behaviour capture devices 1001 (including behaviour capture device 1001a), which are in accordance with the third embodiment of the invention. The behaviour capture system 1000 further comprises a plurality of mobile devices 1002 (including mobile devices 1002a). The behaviour capture system 1000 differs from the previously described behaviour capture systems in that each behaviour capture device 1001 is arranged to be in direct data communication with the server 1003, rather than with a mobile device 602, 902 as shown in FIGS. 6 and 9. The behaviour capture device 1001 and server 1003 are arranged to be in data communication over a wireless network. Preferably, the behaviour capture devices 1001 and server 1003 are arranged to connect using Wi-Fi (®), cellular network, or any other suitable means of wireless communication. The behaviour capture devices 1001 are arranged such that, when connected to the server 1003, the behaviour capture devices 1001 are able to send all data that is stored in the memory module 303, including bookmark data, fidget data, sensor data, location data and all associated data including timestamps, locations and intensities (i.e., behaviour capture data), as well as system data such as the battery level and state of the operating modes of the behaviour capture devices 1001. Furthermore, when connected to the server 1003, the behaviour capture devices 1001 are able to receive data from the server 1003 including: system notifications; instructions to provide feedback; and instructions to change the operating mode of the behaviour capture devices 1001.

The server 1003 is arranged to be in data communication with the behaviour capture devices 1001 over a wireless network as described above. The server 1003 is arranged to store and process data received from the behaviour capture devices 1001. Data received by the server 1003 from the behaviour capture devices 1001 includes all behaviour capture data and system data as previously described. The server 1003 is also arranged to receive and store a complete backup of all behaviour capture data received from the behaviour capture devices 1001.

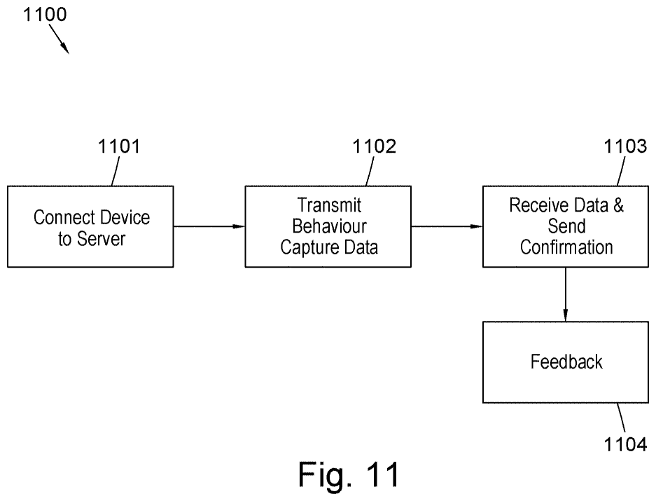

In this arrangement of the behaviour capture system 1000, since the behaviour capture device 1001 is no longer in direct communication with a mobile device 1002, the user is unable to transmit behaviour capture data directly from a behaviour capture device 1000 to the mobile device 1002 as described in FIG. 7. Instead, in the present arrangement of the behaviour capture system 1000, a user may connect a behaviour capture device 1001 to the server 1003 to transmit and store all behaviour capture data on the behaviour capture device 1001 directly to the server 1003, the process for which is shown in FIG. 11. At step 1101, the user first connects the behaviour capture device 1001 to the server 1003 to enable data communication between the behaviour capture device 1001 and the server 1003. Preferably, the behaviour capture device 1001 automatically connects to a wireless network and the server 1003 when in the presence of a known wireless network, for example a Wi-Fi (®) network. Alternatively, the user may configure the behaviour capture device 1001 to connect to and recognise a wireless network.

At step 1102, the behaviour capture device 1001 transmits all new behaviour capture data to the server 1003. This includes the transmission of bookmark and fidget records in the database of the memory module 403, and their respective types, time-stamps, intensities and locations. The transmitted data also includes sensor data stored in the memory module 403. Additionally, the transmitted data also includes location data collected by the behaviour capture device 1001 over time.

At step 1103, the server 1003 receives the transmitted behaviour capture data. Furthermore, upon successfully receiving the behaviour capture data, the server 1003 sends a confirmation message to the behaviour capture device 1001.

At step 1104, the behaviour capture device 1001 receives the confirmation message. Upon receiving the confirmation message, the processor 402 of the behaviour capture device 1001 instructs the feedback module 404 to provide user feedback. Preferably, the feedback pattern exhibited by the feedback module 404 is a pattern that notifies the confirmation to the user. For example, a haptic feedback motor in the feedback module 404 may activate and exhibit a pattern of feedback that notifies the user of confirmation or provides other type of feedback. Alternatively, one or more LEDs in an LED array in the feedback module 404 may blink or light up to notify the user of the feedback or confirmation. It should be noted that the LEDs and haptic feedback motor may exhibit feedback at the same time. Furthermore, preferably, the behaviour capture device 1001 automatically deletes all transmitted data from its memory module 403 upon receiving the confirmation message from the server 1003, in order to make memory space available for subsequent new data entries or records. Alternatively, the behaviour capture device 1001 does not automatically delete the transmitted data in response to the confirmation message, and only deletes the data when instructed to by the server 1003 in a separate instruction.

The user may use the behaviour capture device 1001 for behaviour capture whilst maintaining its connection with the server 1003. In this case, all new behaviour data captured using the behaviour capture device 1001 will be instantly transmitted to the server 1003. Alternatively, the user may disconnect the behaviour capture device 1001 from the server 1003 and continue to use the behaviour capture device 1001 “offline”. In this case, all new behaviour capture data will be maintained on the behaviour capture device 1001 and may be transmitted to the server 1003 at a later time through the process described in FIG. 11.

The server 1003 is also arranged to be in data communication with the mobile devices 1002, similarly to the previous arrangement of the behaviour capture system in FIG. 9. However, in the present arrangement of the behaviour capture system 1000, each mobile device 1002 is unable to receive data directly from a corresponding behaviour capture device 1001 and instead must request and receive behaviour capture data from the server 1003. Since the server 1003 is a centralised location that stores the behaviour capture data of all behaviour capture devices 1001 in the system 1000, security measures and verifications are put in place to ensure that the behaviour capture data sent to a mobile device 1002 from the server 1003 is from the correct corresponding behaviour capture device 1001. Hence, the server 1003 is arranged to transmit behaviour capture data to the mobile devices 1002 upon the verification of a request for such data submitted to the server 1003 by the mobile devices 1002. For example, the server 1003 may receive a request from mobile device 1002a for behaviour capture data captured using the behaviour capture device 1001a, where it is assumed that a user of the behaviour capture device 1001a is also the user of the mobile device 1002a. Hence, the server 1003 is configured to verify that the mobile device 1002a has permissions to access the data stored in the server 1003 that was received from the behaviour capture device 1001a. Preferably, the server's 1003 verification process consists at least one of: registering the behaviour capture device 1001a to a password protected user account from which the data is requested; registering the behaviour capture device 1001a to the mobile device 1002a from which the data is requested; or any other suitable means of data request verification. If the request by the mobile device 1002a is verified successfully, the server 1003 sends the requested behaviour capture data captured using the behaviour capture device 1001a, to the mobile device 1002a.

The mobile devices 1002 otherwise have similar features and associated advantages to the mobile devices described in previous system arrangements. This includes: annotating bookmarks; viewing, analysing and editing the behaviour capture data; changing the behaviours associated with bookmark types; sending all mobile application data to the server to store as a complete backup (e.g., changes to the behaviour capture data, annotations, behaviour assignments to bookmark types and personal information); displaying system information about the behaviour capture device such as battery level and operating mode; issuing notifications, warnings and messages; changing the operating mode of the behaviour capture device; and performing analytics and machine learning for the generation of insights and personalised content. In an alternative embodiment, the server 1003 may perform machine learning and analytics as previously described. Additionally, in the arrangement in FIG. 10, it should be appreciated that interactions between the mobile devices 1002 and the behaviour capture devices 1001 are still possible (such as changing the operating mode of a behaviour capture device 1001), although such communication will instead be via the server 1003.

The behaviour capture system 1000 comprises a user terminal 1004, therapist terminal 1005 and an administrator terminal 1006, the features of which are the same as the features of the corresponding terminals in FIG. 9.

The arrangement of the behaviour capture system in FIG. 10 allows a user of a behaviour capture device 1001 to directly store all behaviour capture data to a server 1003. This has the advantage of making the behaviour capture data more immediately centralised and accessible within the system, without relying on the user to transmit the data first to a mobile device and then a server. For example, behaviour capture data that is directly transmitted to the server 1003 is immediately accessible by a mobile device 1002, user terminal 1004 and therapist terminal 1005, whereas in previous system arrangements, the behaviour capture data is only immediately accessible by a paired mobile device. Additionally, in the case that the server 1003 performs machine learning and analytics on the behaviour capture data, such services are immediately accessible if the behaviour capture data is directly transmitted to the server 1003.

In an alternative embodiment, the behaviour capture device 1001 and mobile device 1002 are also arranged to be in direct communication as described in previous arrangements of the behaviour capture system. Hence, in this alternative embodiment, the user of the behaviour capture device 1001 has the option to communicate and transmit data directly to the server 1003, or to the mobile device 1002.

The following variations of the above embodiments and arrangements will be envisioned within the scope of the invention.

In one variation, the behaviour capture device comprises additional buttons, sensors and switches. Preferably, the additional buttons, sensors and switches are functional to change the operating modes of the device. In a further embodiment, the additional buttons, sensors and switches may additionally or alternatively be functional to record additional types of bookmarks. For example, different types of bookmarks may be recordable using the behaviour capture device by considering the input received from the pressure pad, in combination with different buttons, sensors and switches or with the behaviour capture device.

In another variation, the processor of the behaviour capture device is configured to detect patterns in the sensing data received from the sensor module, such that certain movement patterns of the behaviour capture device are detectable. Preferably, the processor performs functions in response to detecting different movement patterns, such as changing the operating modes of the device. Furthermore, the processor may also use the detectable movement patterns to initiate different types of bookmarks, hence increasing the number of bookmarks detectable by the behaviour capture device.

In another variation, the behaviour capture device further comprises a microphone. Preferably, the microphone is arranged to capture voice. Hence, in this variation, the user may control aspects of the behaviour capture device's functionality using their voice. This may include, for example, recording bookmarks, changing operating modes and initiating data transfers.

In another variation, the user may use peripheral devices in combination with the behaviour capture device. Preferably, the peripheral devices communicate with the behaviour capture device. Alternatively, the peripheral devices may connect to and communicate directly with the mobile device. The peripheral devices are arranged to collect additional data for the purpose of behaviour capture, but external to the behaviour capture device. This may include, for example: a peripheral microphone for capturing voice commands; a ring, wristband, pendant or bracelet for capturing pulse, heartbeat and GSR data; or a pedometer for capturing the number of steps taken by a user. It should be appreciated that in the case that peripheral devices are used to capture physiological data such as pulse, heartbeat and GSR data, the sensor module in the behaviour capture device may not comprise these sensors and instead receive sensing data from these sensors.

In another variation, the mobile application on the mobile device is configured to perform voice recognition. Preferably, the voice recognition is used to navigate the application or provide user annotations to bookmarks.

1. A handheld or wearable behaviour capture device comprising:
a physical input interface configured to receive input from a user related to the user's behaviour, and to generate user input data;
a processor configured to:
receive the user input data;
determine if the user input data corresponds to a plurality of predetermined identifiers; and
generate feedback data; and
a feedback module configured to receive the feedback data from the processor and to provide feedback to the user.

2. A device as claimed in claim 1 and further comprising memory, wherein the processor is further configured to store a bookmark record in the memory in response to determining that the user input data corresponds to one of the predetermined identifiers.

3. A device as claimed in claim 1, wherein the processor is further configured to store a non-bookmark record in the memory in response to determining that the user input data does not correspond to one of the predetermined identifiers.

4. A device as claimed in claim 2, wherein the record includes the predetermined identifier and one or more of a time, a duration, an amount of pressure and a location of the user input.

5. A device as claimed in claim 1, wherein the predetermined identifier is a pattern corresponding to a plurality of distinct user inputs of predetermined duration.

6. A device as claimed in claim 1, wherein the feedback module comprises one or more of a haptic feedback motor, a display and illumination means.

7. A device as claimed in claim 1, wherein the physical input interface is chosen from one or more of a force sensor, a pressure sensor, a push button and a switch configured to detect one or both of a force or a pressure applied to the physical input interface.

8. A device as claimed in claim 1, further comprising sensing means configured to generate sensing data indicative of the orientation and acceleration forces applied to the behaviour capture device.

9. A device as claimed in claim 1, further comprising a GNSS receiver.

10. A device as claimed in claim 1, further comprising a transceiver.

11. A handheld or wearable behaviour capture device comprising:
a resiliently deformable input interface for generating user input data;
a processor configured to:
receive the user input data from the input interface;
generate feedback data; and
a feedback module configured to receive the feedback data from the processor and to provide feedback to the user.

12. A device as claimed in claim 11 and further comprising memory, wherein the processor stores a bookmark record in the memory in response to determining that the user input data corresponds to a predetermined identifier.

13. A device as claimed in claim 11, wherein the processor is further configured to store a non-bookmark record in the memory in response to determining that the user input data does not correspond to a predetermined identifier.

14. A device as claimed in claim 12, wherein the record includes one or more of an amount of pressure, time, duration, intensity and location of the user input.

15. A device as claimed in claim 11, wherein the predetermined identifier corresponds to a plurality of distinct user inputs of predetermined duration.

16. A device as claimed in claim 11, wherein the feedback module comprises one or more of a haptic feedback motor, a display and illumination means.

17. A device as claimed in claims 11, wherein the input interface is chosen from one or more of a force sensor, a pressure sensor, a push button and a switch configured to detect one or both of a force or a pressure applied to the input interface.

18. (canceled)

19. (canceled)

20. (canceled)

21. (canceled)

22. (canceled)

23. A method for behaviour capture comprising:
providing a behaviour capture device that comprises a physical input interface configured to receive input from a user related to the user's behaviour, and to generate user input data; a processor configured to: receive the user input data; determine if the user input data corresponds to a plurality of predetermined indentifers; and generate feedback data; an a feedback module configured to receive the feedback data from the processor and to provide feedback to the user;
receiving at the input interface an input from a user related to the user's behaviour and generating user input data;
receiving the user input data at the processor;
determining at the processor if the user input data corresponds to one of a plurality of predetermined identifiers;
generating at the processor feedback data;
providing the feedback data to the feedback module; and
the feedback module providing feedback to the user.
